(19) United States
(12) Patent Application Publication
(10) Pub. No.: US 2025/0049311 A1
(43) Pub. Date: Feb. 13, 2025

(54) DEVICE, SYSTEM AND METHOD FOR CLEANING AND/OR DRYING AN ENDOSCOPE

(71) Applicant: KARL STORZ SE & Co. KG, Tuttlingen (DE)

(72) Inventors: Sebastian WENZLER, Tuttlingen (DE); Sebastian WAGNER, Tuttlingen (DE)

(73) Assignee: KARL STORZ SE & Co. KG, Tuttlingen (DE)

(21) Appl. No.: 18/722,896

(22) PCT Filed: Dec. 15, 2022
(86) PCT No.: PCT/DE2022/150008

§ 371 Date: Jun. 21, 2024

(30) Foreign Application Priority Data
Dec. 23, 2021 (DE) 10 2021 134 563.4

(51) Int. Cl. A61B 1/12; A61B 1/00; A61B 1/05; A61B 1/06

(52) U.S. Cl. CPC A61B 1/126; A61B 1/00006; A61B 1/00009; A61B 1/00091; A61B 1/00096; A61B 1/05; A61B 1/0676

(57) ABSTRACT

A device for image analysis and/or cleaning of a window arranged at the distal end of an endoscope includes: an image capture device having an optical window for illuminating an object space using an illumination device, a cleaning module including a fluid channel and a nozzle for cleaning and/or drying the optical window by means of a fluid; and a control unit. The control unit analyzes captured image data and, based on a deterioration in the image quality of the captured image data, outputs control instructions for activating image optimization to the cleaning module either automatically or manually so as to activate a fluid pulse for cleaning and/or drying the at least one optical window.

### CROSS-REFERENCE TO RELATED APPLICATIONS

This application is the U.S. national stage of PCT/DE2022/150008 filed on Dec. 15, 2022, which claims priority of German Patent Application No. 10 2021 134 563.4 filed on Dec. 23, 2021, the contents of which are incorporated herein.

### TECHNICAL FIELD

The disclosure relates to a device, a modular system, and a method for cleaning and/or drying an endoscope, in particular at least one optical window arranged at the distal end of the endoscope.

### BACKGROUND

Endoscopes are known medical devices for examining cavities in a body or technical cavities. A commonly used type of endoscope has an optical system at the distal end of the endoscope, i.e. the end facing toward the body, and is designed to capture images and transmit them to the operator of the endoscope. Optionally, further functions can be made available via a working channel.

Endoscopes are known to be used in minimally invasive surgical procedures. An example of this is laparoscopy. Here, the view of the field to be examined is a crucial prerequisite for the operator of the endoscope to be able to carry out a diagnosis, manipulation, or operation safely and quickly. It is important to be able to reliably distinguish between different tissue structures in the body cavity in order to make a correct diagnosis or avoid complications. Optimal visibility conditions are necessary for this purpose.

When intervening in a human or animal body, the field of view of an endoscope is very small and even the smallest contamination, e.g., due to blood splashes, tissue particles, or deposits of steam or smoke or grease, can severely impair the view of the operator. For example, if high-frequency surgery is assisted (HF surgery), thermally induced changes in tissue cells are carried out using electrical energy with the goal of stopping bleeding or sealing tissue. In this type of medical procedure, tissue particles can arise upon the HF activation that severely impair the image for the endoscope operator. If the view of the operator is impaired in this way by unintentional tissue contact or other contamination, the endoscope has to be cleaned extracorporeally.

This extracorporeal cleaning of the endoscope at its distal end is sometimes undesirable, in particular when complications arise and the endoscope operator has to interrupt his medical or diagnostic procedure. In addition, extracorporeal cleaning also requires a certain amount of time, which has to be taken into consideration in the overall duration of an operation. For example, if complications arise due to the perforation of an artery, the bleeding has to be stopped in the shortest possible time and a very good view has to be ensured for the endoscope user. If bleeding cannot be stopped quickly, other surgical methods that involve open surgery may be necessary. This is to be avoided at all costs in order to keep the intervention as minimal as possible for the patient.

A clear and precise visualization of the object field to be observed is of great importance in endoscopic procedures. If visibility is obstructed, the endoscope operator possibly has to interrupt the procedure to clean the endoscope outside the body cavity. Unnecessary interruptions and distractions during the endoscopic procedure can result in misjudgments or endanger the health of a patient into whom the endoscope has been inserted. Any disturbance of the view also prolongs the entire endoscopic procedure, thereby increasing costs and reducing efficiency.

Two known methods for cleaning the endoscope lens are manual wiping or using a lens cleaning system. Manual wiping requires that the endoscope lens or distal optical window be rubbed against nearby soft organ tissue or, as is often the case, that the endoscope be completely withdrawn from the body cavity. To remove tissue particles or bone dust or other contaminants, clean cloths or similar suitable fabrics previously dipped in an anti-fog solution or distilled water are used. Typically, drying is performed before reinserting the endoscope into the body cavity to prepare the optical window for use. However, there is the problem that due to the temperature difference that occurs when the endoscope is inserted into a body cavity, condensation occurs on the optical window, which again impairs the view. Therefore, removal and external cleaning using manual methods is not optimal and requires a large amount of time.

### SUMMARY

Due to the above-mentioned problems, it is the object of the disclosure to meet the needs of endoscopic procedures, wherein a suitable endoscope cleaning system is to have high efficiency and applicability for the user. The operator is to be disturbed as little as possible and, in addition to manual operation, an automatic cleaning method is also to be provided. Furthermore, when providing a modular system, the object is to provide an endoscope having a diameter that does not require more space than the usual endoscope. No special accesses or a trocar are to be necessary for dirt detection and/or for cleaning purposes. There is a need to provide a relatively simply designed and combinable system that is not only easy to handle, but also increases safety during a procedure for the user and patient.

On the basis of the disclosure, the above-mentioned objects are to be achieved better than in conventional devices, in particular for human or veterinary medical applications. The visibility conditions and quality are to be optimized for the operator of the endoscope.

These objects are achieved by a device according to the disclosure, a modular system, and a method for image optimization and/or cleaning and drying of at least one window arranged at the distal end of an endoscope according to the features of the independent claims. Preferred embodiments of the disclosure result from the subclaims following the independent claims.

According to a first aspect of the disclosure, a device for cleaning and/or drying at least one window arranged at the distal end of an endoscope is provided, comprising an image capture device having at least one optical window for capturing image data and/or at least one further window for illuminating an object space using an illumination device, at least one cleaning module comprising at least one fluid channel and at least one nozzle which is designed to clean and/or dry the at least one optical window and/or the at least one further window by means of at least one fluid; and a control unit. The control unit is configured to analyze the captured image data and, based on a deterioration in the image quality of the captured image data, to output control instructions to the cleaning module for activating a fluid pulse for cleaning and/or drying the at least one window automatically or manually by way of an operator and/or at predetermined time intervals, wherein the fluid pulse is adjustable by at least one cleaning parameter by means of the control unit and one or more cleaning parameters is or are selected from a group comprising: pulse duration, number of pulses, pulse-pause ratio, total cleaning duration, type of fluid, fluid volume, fluid volumes, fluid velocity, and/or pressure.

By providing a cleaning module having a fluid channel and a nozzle, the optical window of the endoscope in the body cavity can be cleaned. In this way, external cleaning and interruption of the medical procedure can be avoided. Avoiding interruptions and external cleaning increases safety when using the endoscope. Hygienic safety can also be better ensured than if the endoscope were removed from the body cavity. In addition, a control unit assists the surgeon or the endoscope operator, since with the aid of the control unit, the captured images can be analyzed and a deterioration in image quality can automatically output a control instruction to the cleaning module. If the operator wishes to control the activation of the fluid pulse, the control unit can be configured so that cleaning is only released by manual activation.

The cleaning is adjustable by one or more cleaning parameters, wherein the pulse duration in particular limits the cleaning duration. In other words, liquid is not continuously transported into the body cavity and unnecessary liquid accumulation in the object field to be examined is avoided. Excessive amounts of cleaning liquid could have the opposite effect and impair visibility again or negatively influence the steps to be carried out of an endoscopic procedure. By appropriate and precise setting of the fluid volume, pulse duration, and/or pressure, unwanted bubbles or liquid drops at the end of the endoscope can be prevented. In addition, drying can also be carried out to prevent the windows from fogging up. With a dry optical window and, if necessary, further cleaned windows, a minimally invasive operation can be optimized for the operator and the patient.

According to a preferred embodiment, the at least one nozzle is integrally or detachably connected to the at least one fluid channel and is selected from a group comprising: a flat jet nozzle, a full cone nozzle, a full jet nozzle, and a rotary nozzle.

With the aid of the nozzle variants mentioned, the fluid jet can be directed evenly onto the optical window or other windows. In this way, an optimal cleaning result can be achieved without residual drops in the viewing area. The image quality is improved and the operation risk for the patient is reduced.

A flat jet nozzle, for example, has an impact surface that is either elliptical or rectangular. In this way, an even distribution of liquid and pressure can be achieved for the cleaning processes. This nozzle shape is particularly suitable where an intensive and even jet is required.

The alternative nozzle shape of the full cone nozzle produces a uniform conical jet shape. The droplets produced by the full cone nozzle are relatively large, which allows a high impact force to be achieved, which is an advantage in the case of heavy contamination. Therefore, this nozzle is particularly well suitable for cleaning.

If precise cleaning is required, the full jet nozzle is suitable, which forms a punctiform full jet having increased jet power and high precision. In the case of full jet nozzles, a distinction is made between low-pressure and high-pressure nozzles.

High cone nozzles have a ring-shaped jet shape and are particularly suitable for cooling and dust control, since the jet would not completely hit the lens of the endoscope. If the lens cleaning or window cleaning is to clean the entire distal end of the endoscope, flat jet nozzles, full cone nozzles, full jet nozzles, and/or rotary nozzles are preferably used.

All of the nozzle variants mentioned, such as full cone or hollow cone nozzles, can convey both liquid and gas. After cleaning with liquid, a gas can then be used for drying. If single-channel nozzles are used, the liquid and gas are not separated from one another but are guided in the same channel. The different fluid packets are not supposed to mix, because mixing the liquid with the gas would cloud the liquid and worsen visibility. This makes it all the more important to keep the gas separated from the liquid, for example by way of a specific pulse duration or a suitable pulse-pause ratio.

To avoid mixing of two different fluids, a dual-channel nozzle can be advantageously used. Here, the inlets and the spray channels are separated up to the nozzle outlet.

According to a preferred embodiment, the control unit can set the cleaning parameters for the fluid used in each case so that the pulse duration is at most 3000 milliseconds. In a preferred embodiment, the pulse duration is shorter and is at most 2000 milliseconds.

In this way, a precisely controlled amount of liquid can be dispensed, while a high cleaning speed and a short cleaning time can be ensured. This has the advantage that the operator of the endoscope does not activate the cleaning function for longer than necessary. As a result, not as much liquid accumulates in the body cavity, such as the pneumoperitoneum. Due to the reduction of the liquid, less liquid also has to be suctioned out of the body cavity. With the aid of limiting the cleaning duration, the cleaning time and the cleaning quantity or quantity of liquid can be efficiently automated and coordinated. Such short pulses can only be controlled automatically, since this can only be implemented manually with often undesirable time losses. Since the user no longer has to decide whether and for how long to activate the liquid cleaning function, he can concentrate fully on his actual task. This increases the safety of the patient being treated or diagnosed using the endoscopic procedure.

The cleaning time is designed to be so short that it is not disturbing. It only takes a few seconds and is not in the field of view for a long time or disruptively, as in comparison to windshield wiper cleaning.

According to a preferred embodiment, the control unit is configured to set the cleaning parameters for the fluid used in each case depending on the nozzle geometry used in each case such that the pulse duration is at most 3000 milliseconds, preferably at most 2000 milliseconds. A distinction is to be made between the total cleaning time and the pulse duration of the individual cleaning pulse.

With the aid of the control unit, the connected optics and/or the connected cleaning module and nozzle geometry (e.g. single-channel or dual-channel) can be recognized by the control unit on the basis of an initialization routine. Depending on the nozzle geometry used and the optics recognized in each case, a suitable pulse duration can be selected.

In known cleaning procedures that enable in situ cleaning using distilled water or a physiologically harmless and biocompatible liquid, such as sodium chloride solution, at the distal end of the endoscope, the liquid flows through a flushing channel and then down the endoscope lens according to the waterfall principle. The problem here, however, is that the cleaning function is only partially targeted and heavy soiling cannot always be removed. Therefore, conventional cleaning times using the waterfall principle are have a duration of approximately 5 seconds or more. By means of a suitable nozzle geometry and a short pulse duration having a higher pressure and strength, the optical window or, if necessary, other windows can be cleaned optimally and efficiently in significantly shorter cleaning durations than 5 seconds.

According to a preferred embodiment, the fluid channel is designed such that a fluid channel diameter (D) is equal to or up to a maximum of 20% larger than the nozzle cross section, wherein the nozzle is preferably a flat jet nozzle. Furthermore, the cleaning module is connectable to a device for generating pressure or a pressure line in order to direct a closed fluid jet, which is preferably fan-shaped and flat, under high pressure onto the optical window and/or the further window for cleaning and/or drying after activation of the fluid pulse of the cleaning module.

With the aid of a device for pressure generation or a pressure line, a fluid jet can be directed with high pressure at the optical window or another window. With the aid of pressure, drying can also take place faster and more efficiently. Due to the more efficient cleaning, a smaller flushing quantity of gas or fluid is necessary and the required cleaning flushing quantity can be reduced to a minimum.

In contrast to the pressure-controlled cleaning method according to the present disclosure, conventional methods based on the waterfall principle for cleaning can only work without pressure. The known waterfall technique is therefore much less targeted than a closed fluid jet, which is preferably fan-shaped and flat.

Another known problem results in a configuration when two channels are provided to avoid mixing of two different fluids. In this dual-channel configuration, the inlets and the flushing channels are spatially separated up to the nozzle outlet, so that the technical problem arises that the nozzle opening can no longer be located centrally and the fluid jet hits the optical window or other windows with an offset. If a nozzle such as a flat jet nozzle or full cone nozzle is selected according to the disclosure, all relevant points of the viewing window or another window, such as a window for the illumination outlet, can be covered by a suitable choice of a larger opening angle of the respective nozzle in comparison to the opening angle of a single-channel nozzle.

Advantageously, physical laws such as the continuity law or the Venturi effect can be used when choosing the geometric dimensions of the nozzle and the fluid channel. It is known that the flow velocity can be increased by narrowing a fluid channel diameter and/or reducing the cross-section in a nozzle. Advantageously, the nozzle cross-section can be selected to be smaller than the fluid channel diameter so that the fluid velocity increases and can be used for efficient cleaning.

For endoscopic procedures, it is also advantageous that the nozzles and the adjacent supplying fluid channel have a relatively small diameter so that the outer diameter of the overall system, i.e., the endoscope together with the cleaning system, does not increase significantly. Ideally, the outer diameter of the overall system is not increased in comparison to a system without a cleaning system, so that a standard trocar access can be selected.

According to a preferred embodiment, the fluid comprises a liquid, wherein the liquid fluid volume or fluid volumes predetermined for cleaning is or are less than or equal to 5 mL, preferably less than 3 mL. There is preferably a pressure in a fluid supply line of at least 0.5 bar, preferably 2.5 bar.

According to a preferred embodiment, the liquid fluid is a physiologically safe and biocompatible liquid, preferably a physiological saline solution.

Medically approved cleaning liquids such as purified water or sterile physiological saline solution can safely be brought into contact with objects to be examined in the body cavity of the patient.

According to a preferred embodiment, the fluid is liquid and/or gaseous and the cleaning is controllable using multiple fluid pulses having a duration of a few milliseconds up to a maximum of 1000 ms, preferably having a duration in a range of 200-800 ms. Narrower ranges are settable. These short fluid pulses are only settable accurately and precisely by an automatic controller. The results of cleaning and/or drying can be monitored by means of a software-based monitoring routine based on image data evaluation of the field of view. For this purpose, parameters are stored in a memory that can be used to classify a cleaning result as positive or negative. If the cleaning result is not yet sufficient and the target values are not met, another fluid pulse is automatically activated for cleaning and/or drying until the cleaning result is positive or corresponds to the target values.

According to a preferred embodiment, the fluid is gaseous, wherein the fluid velocity of the gaseous fluid volume or volumes is less than 15 centiliters per second and the maximum pressure in the fluid supply line is 3 bar.

According to a preferred embodiment, the gaseous fluid is physiologically harmless and biocompatible, preferably carbon dioxide. All gases approved as medical products, for example those approved for use in conventional insufflators, can be used for this purpose. For example, carbon dioxide is to have a purity of preferably 99% and a maximum moisture content of 25 ppm

According to a preferred embodiment, the cleaning using the gaseous fluid is controllable by the control unit continuously using fluid pulses at intervals each having a duration of a maximum of 1000 ms or continuously.

According to a preferred embodiment, a fluid outside the cleaning module can be conveyed by at least one pumping device or a gas source by means of a supply line into the body cavity and through a discharge line out of the body cavity and the device furthermore comprises a pressure sensor for measuring the intracorporeal pressure, wherein the control unit controls the intracorporeal pressure in an event-controlled and/or time-controlled manner at least during the duration of a cleaning by means of a control of the at least one pumping device or a control of a pressure regulator such that the intracorporeal pressure does not exceed a predetermined maximum limiting value.

By measuring the intracorporeal pressure in the body cavity, the pressure can be reliably monitored in order to automatically compensate for overpressure and reliably protect against excessively high pressures. In addition to the automatic control to avoid overpressure, a pressure relief valve is also to be provided.

According to a preferred embodiment, the nozzle is fixedly positionable at a predetermined distance relative to the optical window and/or further window, so that the fluid jet is directed over the entire external geometry of the optical window and/or the further window.

According to a preferred embodiment, the optical window and/or further windows are formed by an at least partially convex surface.

By providing a convex surface of the cover glass of a window or a similar distal geometry, the Coanda effect can advantageously be used. In other words, the gas jet or a liquid jet from the nozzle runs along the convex surface and does not detach prematurely, thus ensuring complete cleaning of the window

According to a preferred embodiment, the optical window is an endoscope and the at least one illumination device comprises light-conducting fiber bundles, LEDs (light emitting diodes), OLEDs (organic LEDs), one or more other light sources, or combinations thereof. The illumination device and/or the cleaning module is integrally and/or detachably connected to the endoscope, wherein the endoscope is selected from a group comprising the following image capture devices: a camera, an optoelectronic recording system, a digital camera, a CMOS image sensor, or a CCD image sensor.

The illumination device can also cause the optical window to heat up. Lens heating can also be controlled by the control unit by means of activating the cleaning module. In this way, the cleaning module can be used as an endoscope cooler.

Preferably, the at least one further window is assignable to the illumination device. Simultaneous cleaning of the optical window and the illumination window improves the light output and also ensures a longer service life of the illumination device.

According to a preferred embodiment, the at least one image capture device having at least one illumination device is designed to be insertable into a shaft and replaceable. Furthermore, the control unit preferably has a memory and a processor for image recognition and optics recognition, preferably with the aid of an initialization routine, in order to recognize the image capture device and to transmit stored cleaning parameters depending on the recognized image capture device to the device for cleaning activation.

In this way, the image capture device can be easily exchanged and optimally adapted to the different procedures such as arthroscopy or laparoscopy. Advantageously, the various usable image capture devices can be stored in a memory and the optimal cleaning parameters can be preset by means of automatic optics recognition.

According to a preferred embodiment, the cleaning module is part of a kit. The kit comprises: a pressure sensor for measuring the intracorporeal pressure and/or at least one further illumination device having an identical or modified orientation as the illumination device of the image acquisition device.

A pressure sensor can advantageously be used to ensure that the maximum pressure in the body cavity to be examined is not exceeded. In this way, patient safety can be ensured, for example during insufflation with CO2.

According to a preferred embodiment, the kit comprises at least one partially circular or oval outer sleeve and/or a shaft. A round or oval outer geometry is suitable to ensure an optimal trocar seal.

According to a preferred embodiment, a modular system comprises a device for cleaning and/or drying at least one window arranged at the distal end of an endoscope having at least the features according to the independent device claim. The modular system furthermore comprises a shaft having a proximal and a distal end and at least one receptacle for at least one further component, wherein the component is selected from the following group: at least one working channel extending through the shaft from proximal to distal; and/or; at least one suction channel extending through the shaft; at least one flushing channel extending through the shaft; at least one detachable distal sealing unit for sealing a fluid channel or a suction channel: at least one fixed or detachable coupling to the image capture device: at least one distal outlet or inlet which is aligned in the direction of the distal end or another predetermined object; and combinations thereof.

According to a preferred embodiment, the cleaning module and/or the components are reprocessable, sterilizable, or designed as disposable items.

According to a preferred embodiment, the cleaning module and/or the components are detachably or integrally connected to another component and/or the image capture device.

According to a preferred embodiment, the outer diameter of the shaft is configured such that the image capture device having at least one illumination device and having the cleaning module is receivable by an access system to the body cavity, preferably by a trocar sleeve.

In this way, standard dimensions of trocars can be used.

According to a further aspect of the disclosure, a method for image analysis and/or cleaning of at least one window arranged at the distal end of an endoscope is provided, comprising the following method steps:
providing an optical window of an image capture device and/or a further window for illuminating an object space using an illumination device and preferably at least one cleaning module;
capturing image data by means of the image capture device;
analyzing the image data by way of a control unit to detect a deterioration of the image quality, and on the basis of the detected image quality, automatically by means of a self-learning module and/or manually by the operator, outputting control instructions from the control unit to a unit of the endoscope to activate image optimization.

For example, an artificial intelligence or a neural network can be provided as a self-learning module. The self-learning module preferably has a trained model based on machine learning or deep learning. By analyzing the image data and by way of suitable software-based algorithms of the self-learning module, automatic image recognition can be carried out and, for example, contamination in the field of view can be detected.

According to a preferred embodiment, the method for image optimization comprises cleaning at least one optical window arranged at the distal end of an endoscope, wherein, on the basis of the method step of analyzing the image data and based on a deterioration in the image quality of the acquired image data, control instructions are output automatically or manually by an operator and/or at predetermined time intervals from the control unit to the cleaning module for activating a fluid pulse for cleaning and/or drying the optical window and/or the further window for image optimization, and wherein the control unit sets at least one cleaning parameter which is selected from the group comprising: pulse duration, number of pulses, pulse-pause ratio, total cleaning duration, type of fluid, fluid volume, fluid volumes, fluid velocity, and/or pressure.

With the aid of these method steps, an automated overall system can be provided which provides automatic dirt detection by way of digital image analysis and, depending on the image quality, deliberately cleans the at least one optical window within a short time, preferably with a short cleaning time of a few seconds, preferably at most 3000 ms. Due to the short cleaning time, the operator is not disturbed during the endoscopic procedure, like a windshield wiper. This can simultaneously improve image quality and reduce the operation risk. Such short cleaning times in combination with high pressures of up to 1 bar for liquids are only controllable in a defined and precise manner automatically by software.

### BRIEF DESCRIPTION OF DRAWINGS

The disclosure as well as further advantageous embodiments and developments thereof are described and explained in more detail below with reference to the examples shown in the drawings. The drawings are for illustration purposes only and are not to scale. Terms such as top, bottom, above, and below are not to be understood as restrictive. The features shown in the following description and the drawings can be used according to the disclosure individually or in groups in any combination.

FIG. 1a is a perspective view of a distal end of an endoscope having a cleaning device according to the disclosure:

FIG. 1b shows an exploded view and a detailed view of FIG. 1a, wherein the nozzle and two fluid channels of the cleaning module are shown;

FIG. 1c shows an embodiment of an optical window which is convexly shaped;

FIG. 2 shows a schematic representation of the device or the system for cleaning:

FIG. 3a shows another embodiment of the device for cleaning:

FIG. 3b shows an exploded view of FIG. 3a: the shaft with the cleaning module is shown separately from the imaging system or endoscope:

FIG. 4a shows another embodiment of the cleaning module having an imaging system and shaft:

FIG. 4b shows the cleaning nozzle separately from the two fluid channels, as well as a detailed view of the distal end of the illumination device:

FIG. 5a shows a further embodiment of the device, wherein only one fluid channel is provided:

FIG. 5b shows the embodiment of FIG. 5a, wherein the imaging system is displaced with respect to the front distal end for better illustration:

FIG. 5c shows the device having the shaft and the cleaning module, which is designed as single-channel:

FIG. 6a shows another embodiment of a single-channel cleaning device:

FIG. 6b shows another embodiment of a single-channel cleaning device having an elongated hole:

FIG. 6c shows a dual-channel cleaning device:

FIG. 7a shows an embodiment of a cleaning nozzle which is designed as a flat jet nozzle and is shown in cross section:

FIG. 7b shows section position a-a of FIG. 7a and thus a detailed view along the slot:

FIG. 8 shows a flow chart of the cleaning method according to the disclosure;

FIG. 9 shows a schematic flow chart of the input parameters for the cleaning parameters for a liquid fluid and a gaseous fluid:

FIG. 10 shows a flow chart to schematically represent the process monitoring.

### DETAILED DESCRIPTION OF PREFERRED EMBODIMENTS

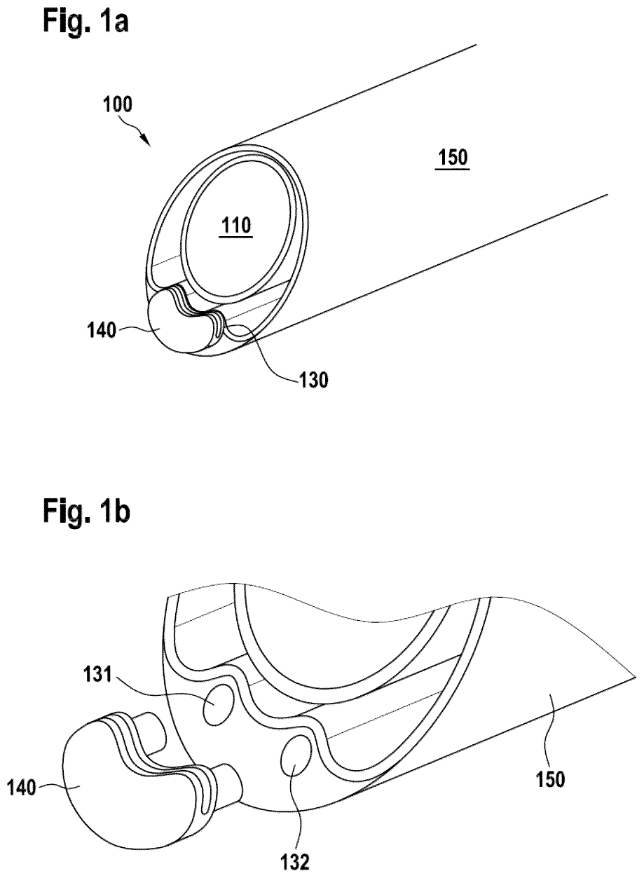

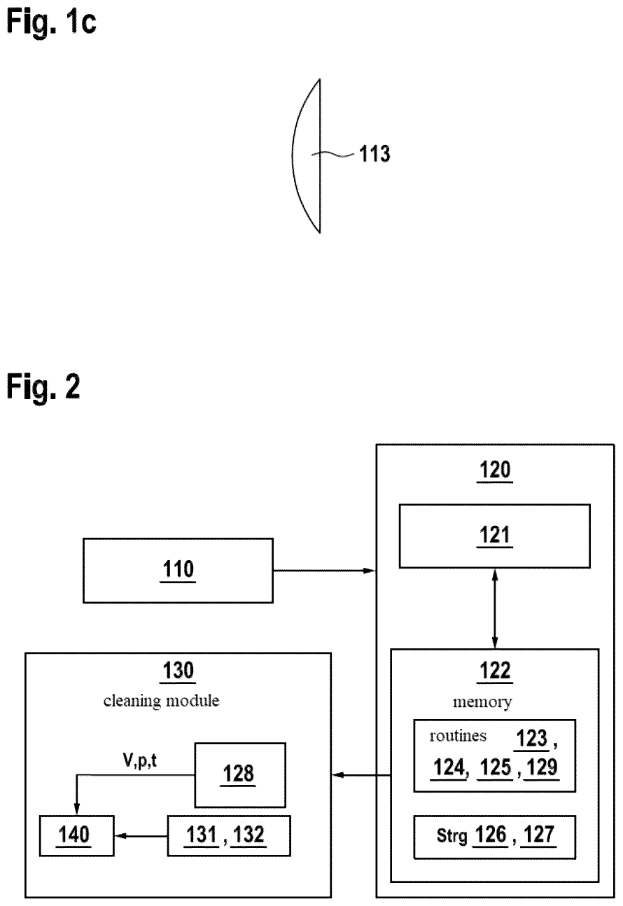

FIG. 1a is a perspective view of a distal end of an endoscope having a cleaning device according to the disclosure, wherein FIG. 1b shows a detailed exploded view of FIG. 1a. The embodiment according to FIG. 1 has a cleaning module 130 having a nozzle 140. The optical window 110 of an image capture device such as an endoscope is arranged above the cleaning module 130 in a common shaft 150.

The components shown show a modular system 100, wherein the cleaning module 130 is detachably or integrally connectable to the other components and/or the image capture device.

FIG. 1b shows that the cleaning module 130 has two fluid channels 131, 132. In this way, the cleaning fluids can be transported separately from one another to the optical window 110. To avoid mixing of two different fluids, in this embodiment a dual-channel nozzle is advantageously used as the nozzle 140, wherein the inlets and the spray channels extend separately up to the nozzle outlet.

FIG. 1c shows a convex surface 113 of an optical window or a cover glass of another window. By providing a convex surface 113 of the cover glass of a window or a similar distal geometry, the Coanda effect can advantageously be used. In other words, the gas jet or a liquid jet from the nozzle runs along the convex surface and does not detach prematurely, thus ensuring complete cleaning of the entire window surface.

FIG. 2 shows a schematic representation of an embodiment according to the disclosure of a device or a system for cleaning and/or drying at least one optical window at a distal end of an endoscope. The cleaning system comprises an image sensor having an optical window 110, a control unit 120, and a cleaning module 130. The image data captured by the image sensor are transmitted to the control unit 120 for further processing by the processor 121 and possible storage of the image data in a memory 122.

The processor 121 can comprise one or more microprocessors and can be used for image analysis 124 and optics recognition 123. The optics recognition 123 can be used as an initialization routine to recognize the connected image capture device and to transmit stored cleaning parameters 128 depending on the recognized image capture device to the cleaning module 130 for the cleaning control or activation. Cleaning parameters 128 (fluid volume V, pressure p, and pulse or cleaning duration t) are stored in the memory for the respective cleaning module 130. The cleaning parameters can comprise pressure p, number of pulses, pulse duration t, total cleaning duration t, pulse-pause ratio, type of fluid, fluid volume V, fluid volumes (liquid or gas quantity), and/or fluid velocity.

With the aid of the cleaning parameters 128, the pressure p and the pulse duration t of a fluid pulse or a plurality of pulses in the cleaning module 140 can preferably be optimally controlled. FIG. 2 shows an exemplary embodiment having two separate fluid channels 131, 132. Other cleaning modules 140 not shown here, for example having only one fluid channel 131 that can convey both liquid and gas in one channel, can also be controlled using the control unit 121 and the system.

The routines in addition to optics recognition (initialization routine 123) and analysis 124 of the image data to detect a deterioration in image quality (image recognition) comprise at least one or more of the following additional routines:
Evaluation routine or classification routine 125, whether image soiling was detected,
control (abbreviated as strg in FIG. 2) of the cleaning 126, preferably by liquid, and/or control of the drying 127, and
monitoring routine 129, whether the cleaning was sufficiently successful.

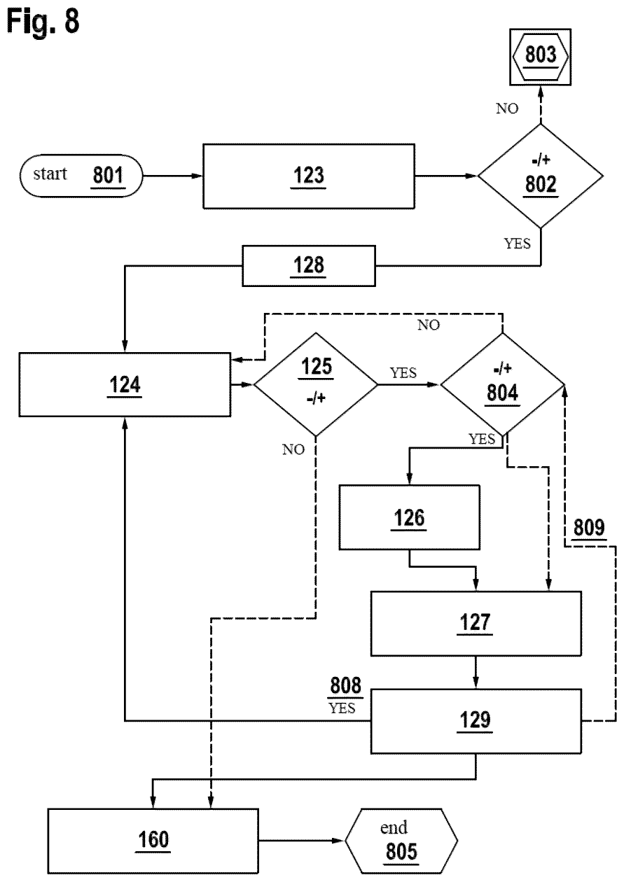

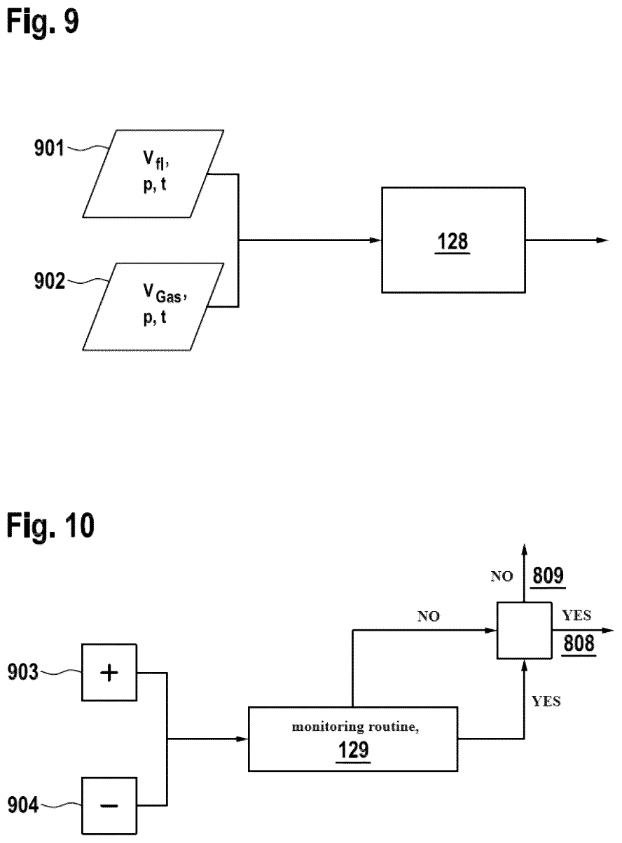

Further details on the control routines and method steps are explained in FIG. 8, FIG. 9, and FIG. 10.

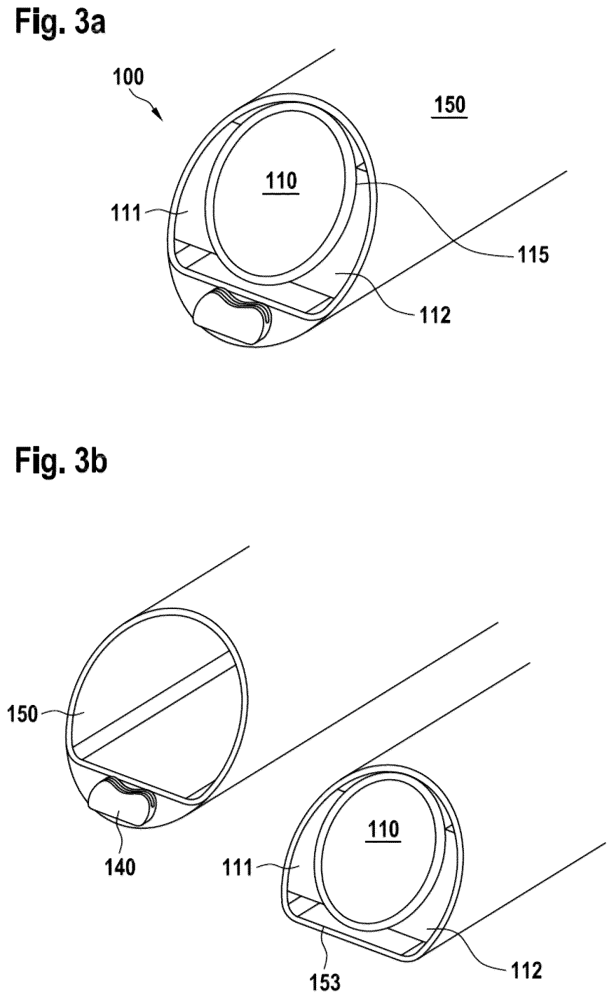

FIG. 3a to FIG. 3b show a modular system 100. The modular system 100 consists of the optical window 110 of an image capture device, preferably an endoscope, as well as two illumination devices 111 and 112 arranged laterally to the optical window. The components are arranged in the shaft 150.

FIG. 3b shows on the left side the shaft 150 having the nozzle 140 for cleaning using a fluid in the lower area. A unit is shown adjacent thereto, which is surrounded by a sleeve 153 and can be inserted into the shaft. This unit comprises the imaging system endoscope having the optical window 110 in the center and illumination devices 111 and 112 arranged on both sides, wherein each illumination device is arranged between the optical window 110 or the endoscope and the sleeve 153. The light can be supplied using glass fibers or other suitable means.

In addition, FIG. 3a illustrates that various inserts having endoscope and one or more illumination devices 111, 112 can be introduced into the shaft 150 with the cleaning nozzle 140, as long as the outer sleeve of the sleeve 153 is smaller than the inner diameter of the shaft 150. A preferred embodiment of a sleeve 153 is shown in FIG. 3b, wherein the lower area is cut off to provide sufficient space in the shaft 150 for the unit of the cleaning nozzle 140. The diameter of an endoscope or the optical window 110 can be approximately 10 mm or less. The sleeve 153 forms a closed outer sleeve around the imaging system, in particular around the endoscope together with the illumination devices 111 and 112.

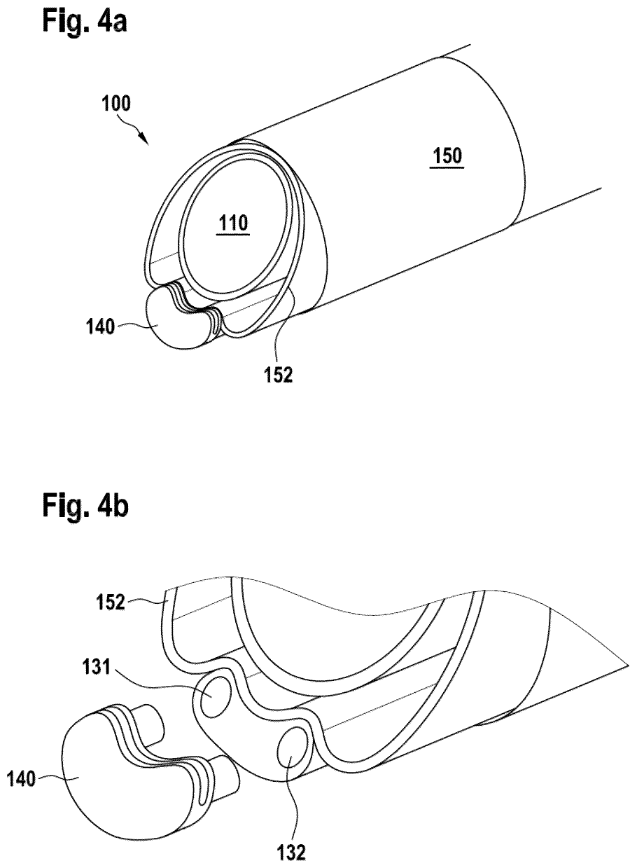

Preferably, the optical window 110 is oval or circular and the illumination devices 111, 112 adapt to both the outer shape of the optical window 110 and the inner shape of the sleeve 153. Thus, the outer shape of the sleeve 153 also results in an at least partially circular contour or arced geometry, wherein the lower side can be cut straight to form essentially a D-shaped cross section of the sleeve 153. The sleeve 153 having D-cross section can also have other suitable geometries depending on the shape of the cleaning unit and an imaging system, for example as shown in following FIG. 4a.

Optionally, a part of the receptacle or the border 115 of the optical window can be blackened. This avoids unwanted reflections on the corresponding surfaces, so that the image quality can be increased.

FIG. 4a and FIG. 4b show another preferred embodiment of a sleeve 152 around the imaging system, which is configured for a dual-channel nozzle 140. This sleeve 152 has a semi-circular upper cross section and adapts in the lower area to the outer contour of the two channels 131, 132. This results in a wave shape in the lower area of the sleeve 152. FIG. 4b shows that the nozzle 140 has two access channels to the respective channels 131, 132. In FIG. 4a, this cleaning nozzle 140 is in the mounted or attached state and provides a fluid-tight connection.

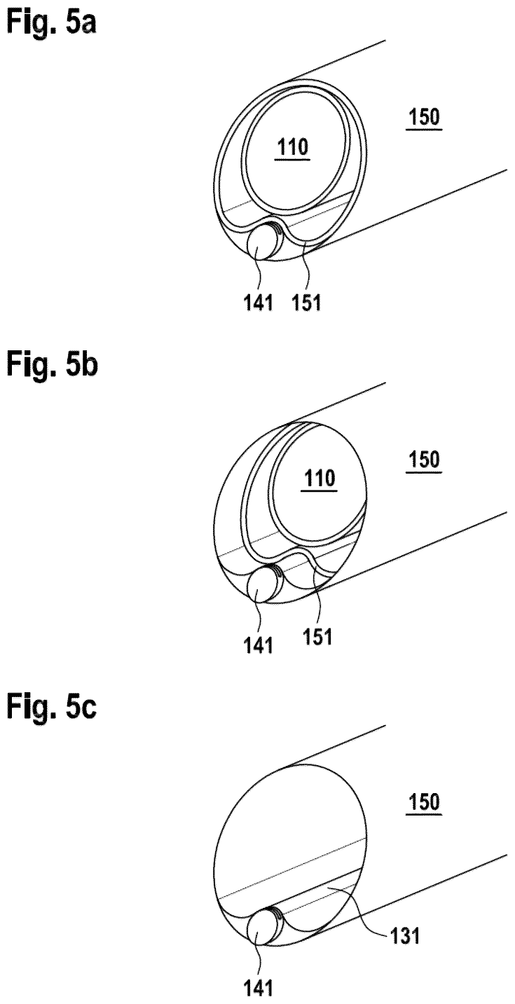

FIGS. 5a to 5c show overall and exploded views of an embodiment having a single channel nozzle 141 having a single supply channel 131 (indicated in FIG. 5c). The sleeve 151 also forms an intermediate sleeve between the shaft 150 and the imaging system having an illumination device not shown here. The imaging system has an optical window 110. The sleeve 151 conforms in the lower area to the individual channel 131 having a convex or concave shape in a wave-like manner.

It is illustrated in FIGS. 5a to 5c that the individual components, such as sleeve 151 and optical window 110, can be introduced into the shaft 150 with the associated endoscope. FIG. 5a and FIG. 5b show different positionings of the imaging system in relation to the nozzle. This flexible design allows tolerances to be compensated. Once the correct position is found, the sleeve 151 and the cleaning nozzle 141 can be fastened on the endoscope. In this way, the distance between the nozzle 141 and the optical window 110 can be ensured exactly.

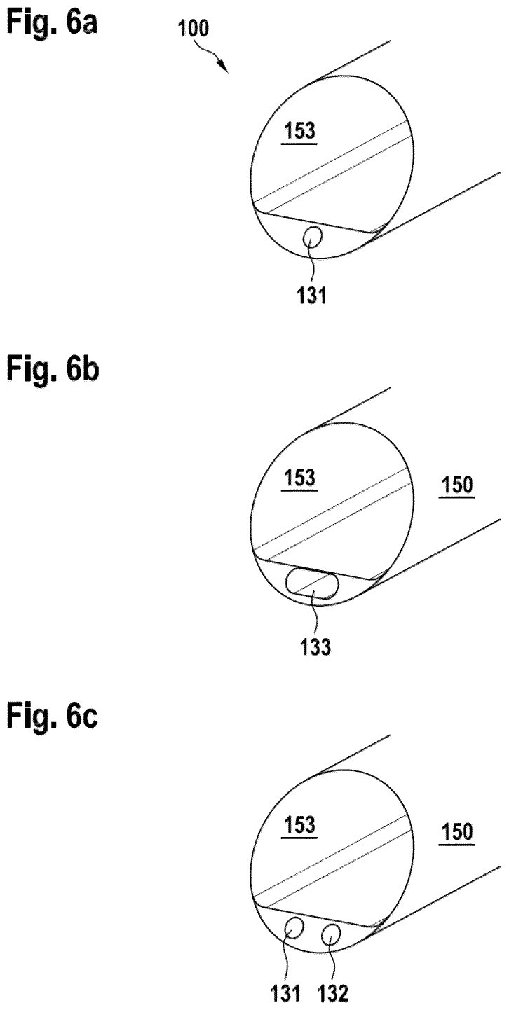

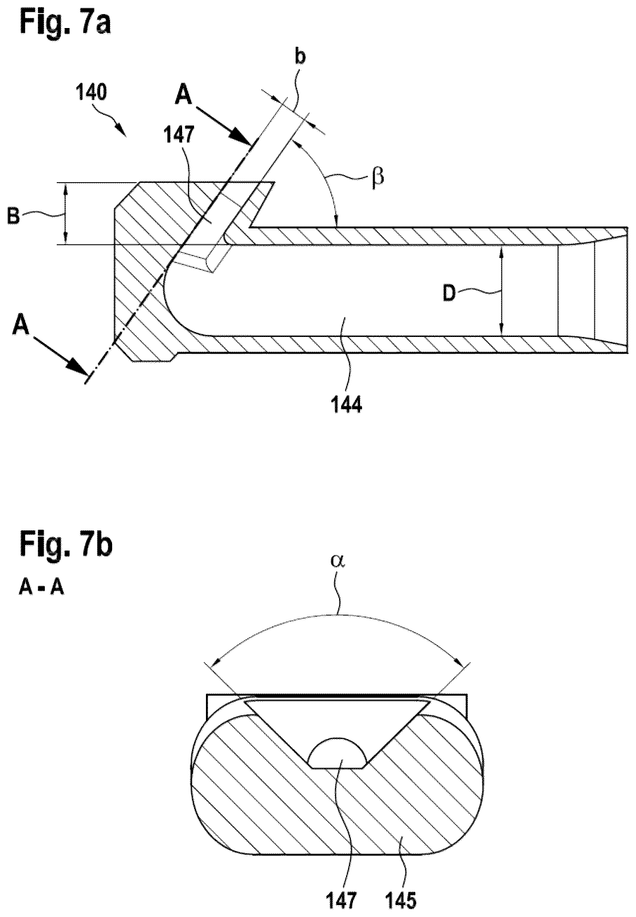

FIG. 6a shows an embodiment having a single channel 131. The sleeve 153 is D-shaped to provide sufficient receiving space for the one channel 131 in the shaft 150. The sleeve 153 also has in FIG. 6b and FIG. 6c essentially a D-geometry having rounded corners. Furthermore, these modular systems can have a pressure measuring probe for measuring the intracorporeal pressure (not shown here). The embodiments of the cleaning unit differ in FIGS. 6b and 6c.

According to FIG. 6b, a wider feed channel 133 is provided in the form of an elongated hole. This elongated hole 133 is adjoined by a single channel along the shaft. A flushing nozzle not shown here can be connected to the elongated hole.

FIG. 6c shows a modular system having two channels 131 and 132. One illumination unit or multiple illumination units (not shown) can also be positioned here at the end of the sleeve 153 together with an endoscope. In this embodiment it is also possible to provide a pressure measuring probe for measuring the intracorporeal pressure.

FIG. 7a shows a cross-sectional view of a cleaning nozzle designed as a flat jet nozzle. The cross section D of the elongated fluid channel 144 decreases in the angled spray channel to the narrower cross section b, which is arranged in the spray channel wall B of the nozzle. According to the Venturi effect, the speed increases in the narrower area. The spray channel is inclined at an angle β with respect to the longitudinal axis of the feed channel 144, so that a liquid jet emerges from the nozzle opening at an angle β.

Section A-A in FIG. 7b shows a section along the spray channel 147, as indicated in FIG. 7a by A-A and the corresponding arrows. FIG. 7b shows the opening angle α of the nozzle and the wall 145. The opening angle α of the cross section of the spray channel 147 is selected such that the optical window to be cleaned is completely cleaned. This angle α depends on the endoscope used and optionally also on other windows of the illumination devices used and the angle α shown is only an example. The expansion of the cross section increases the pressure of the emerging liquid. The fluid used for cleaning or drying can be deliberately directed at high pressures onto the window to be cleaned. Preferred pressure ranges and limiting parameters are summarized for a single-channel or dual-channel nozzle in following Table 1:

TABLE 1
Limiting parameters of the cleaning concept

| | | Limiting parameters | | | |
|---|---|---|---|---|---|
| | Nozzle type | One channel | | Two channel | |
| Physical | Liquid pressure | 0.01-1.0 | bar | ≥0.5 | bar |
| parameters | liquid volume | 0.4-3.0 | bar | 0.6-3 | ml |
| | Gas pressure | 0.5-5.0 | bar | 0.5-5.0 | bar |
| | Flushing time | ≤200 | ms | ≥200 | ms |
| | Drying time | ≥1 | s | ≤0.4 | s |

| | | |
|---|---|---|
| Nozzle | Opening angle | strongly dependent on the endoscope |
| parameters | Wall | ≥0.20 mm |
| | Exit angle α | strongly dependent on the endoscope |
| | Diameter | >0.30 mm |
| | Slot width | ≥0.10 mm |
| | Fluid channel diameter | ≥Nozzle diameter |

FIG. 8 schematically shows the method steps of the method according to the disclosure for image analysis and/or cleaning of at least one distal window.

As a first step, an image acquisition device having an optical window is provided. In step 801, the system controller starts suitable software or a program to first execute an initialization routine 123. As part of the initialization routine 123, an image and/or optics recognition is started. The software uses the image to recognize which optics are connected. For example, the diameter of the optical window used can be determined, which can be in the range between 5 and 10 mm. Furthermore, the optics recognition can determine with the aid of the initialization routine 123 whether a rigid or a flexible endoscope is used and which orientation (0°, 30°, 45° and values in between) of the endoscope and/or the exit angle of the cleaning nozzle are used.

In the case that no optical unit is recognized in step 802, which can comprise an image evaluation step (marked “NO” on the flow chart), the process is aborted in step 803 and it is reported to the operator that no optical unit has been recognized. In this case, the connections to the control unit should be checked and/or whether all components are supplied with power. If all components were switched on, a different optical unit may be used.

In the case that in step 802, an optical unit is detected (marked “YES” on the arrow), the method is continued. Depending on the determined endoscope parameters, the appropriate cleaning parameters 128 are preselected for the cleaning module. This can be done either automatically on the basis of the initialization routine 123 or by a user. As shown in FIG. 2, the cleaning parameters include 128 parameters such as required fluid quantity V, pressure p, and time t.

The analysis routine 124 is started as the next method step. In this analysis 124, the image data are evaluated by a preferably self-learning algorithm using an artificial intelligence. A comparison of the currently captured image data with previously stored image data can enable automatic detection of the operating room environment and/or contamination of the optical window or field of view.

In step 125, it is first checked whether the captured image shows a body cavity to be examined, intracorporeal structures, or an operating room environment. If this query has a negative result (“NO” for dashed arrow), the process is aborted in step 160 and the method is ended in step 805 (“End”).

If this query has a positive result (“NO” for dashed arrow), it is checked in step 804 whether or not the optical window has been contaminated. If no contamination is detected, the analysis routine 124 can be continued with current image data.

If contamination is established in step 804, cleaning 126 is carried out depending on the degree of soiling. If severe contamination is established, cleaning 126 with liquid is necessary, while in the case of light contamination or fogging of the optical window, cleaning with gas may be sufficient and the control system activates drying 127. The previously preselected cleaning parameters are used for the selected cleaning 126 or drying 127. The input data for the cleaning parameters are shown in detail in FIG. 9.

Furthermore, the results of the cleaning 126 and/or drying 127 can be monitored by means of a monitoring routine 129. For this purpose, parameters are stored in a memory that can be used to classify a cleaning result as positive or negative. Details of the monitoring routine are schematically shown in FIG. 10. If the monitoring routine 129 indicates that the field of view and image quality is again optimal (“YES”), the analysis routine 124 is performed. If the cleaning result is not optimal, the routine 804 for dirt detection is called (see arrow 809) and then cleaning 126 and/or drying 127 can be carried out again.

FIG. 9 shows cleaning parameters 128 for a liquid 901 comprising volumes V_{Fl}, pressure p, and time p or for a gas 902 V_{Gas}, pressure p, and time t.

FIG. 10 shows that either a positive cleaning result 903 (see also as output “YES” arrow) or a negative cleaning result 904 (see also as output “NO” arrow) can be established by the monitoring routine. In case of a negative result, the process is referred back to the contamination detection 804 according to 809. In case of a positive result, the image analysis 124 is performed again (see arrow 808 in FIG. 10 or FIG. 8).

With the aid of the monitoring routine, safety can be increased for the patient. The automatic dirt detection in step 908 can quickly activate one or more cleaning and/or drying processes 126, 127. The monitoring routine 129 provides a closed control loop that checks whether or not predetermined target values of the image quality are achieved by the activated cleaning (cleaning 126 by means of liquid or drying 127 by means of gas). The cleaning quality can thus be ensured. By selecting very short fluid pulses that last a maximum of a few seconds or milliseconds, the user is not disturbed by the cleaning process. Rather, the cleaning pulses of single-channel nozzles with a duration of less than or equal to 200 ms are barely noticeable to the user in the field of vision, like a fast-moving windshield wiper.

1. A device for cleaning and/or drying at least one window arranged at the distal end of an endoscope comprising:
an image capture device having at least one optical window for capturing image data and/or at least one further window for illuminating an object space using an illumination device,
at least one cleaning module comprising at least one fluid channel and at least one nozzle which is designed to clean and/or dry the at least one optical window and/or the at least one further window by means of at least one fluid; and
a control unit,
wherein the control unit is configured to analyze the captured image data and, based on a deterioration in the image quality of the captured image data, to output control instructions to the cleaning module automatically or manually by an operator and/or at predetermined time intervals to activate a fluid pulse for cleaning and/or drying the at least one optical window,
wherein the fluid pulse is adjustable by at least one cleaning parameter by means of the control unit, wherein the at least one cleaning parameter is selected from a group comprising:
a pulse duration, a number of pulses, a pulse-pause ratio, a total cleaning duration, a type of fluid, a fluid volume, a fluid volumes, a fluid velocity, and/or a pressure.

2. The device according to claim 1, wherein the at least one nozzle is integrally or detachably connected to the at least one fluid channel and is selected from a group comprising:
a flat jet nozzle, a full cone nozzle, a full jet nozzle, and a rotary nozzle.

3. The device according to claim 1, wherein the cleaning parameters for the fluid used in each case are adjustable by means of the control unit such that the pulse duration is at most 3000 milliseconds (ms), preferably at most 2000 ms.

4. The device according to claim 1, wherein the cleaning parameters for the respective fluid used and depending on the respective nozzle geometry used can be adjusted by means of the control unit such that the total cleaning time is less than or equal to 3000 milliseconds (ms), preferably 2000 ms.

5. The device according to the claim 2, wherein a fluid channel diameter is the same size or up to at most 20% larger than the nozzle cross section, preferably of a flat jet nozzle, and the cleaning module is connectable to a device for generating pressure or a pressure line in order to direct a closed fluid jet, which is preferably fan-shaped and flat, under high pressure onto the optical window and/or the further window for cleaning and/or drying after activation of the fluid pulse of the cleaning module.

6. The device according to claim 1, wherein the fluid comprises a liquid and the liquid fluid volume or fluid volumes predetermined for cleaning is or are less than 5 mL, preferably less than 3 mL, and the pressure in a fluid supply line is at least 0.5 bar, preferably 2.5 bar.

7. The device according to claim 6, wherein the liquid fluid is a physiologically safe and biocompatible liquid, preferably a physiological saline solution.

8. The device according to claim 1, wherein the fluid is liquid and/or gaseous and the cleaning can be controlled with several fluid pulses with a duration of a few milliseconds up to at most 1000 ms, preferably with a duration in a range of 200-800 ms.

9. The device according to claim 1, wherein the fluid is gaseous, wherein the fluid velocity of the gaseous fluid volume or volumes is less than 15 centiliters per second and the maximum pressure in the fluid supply line is 3 bar.

10. The device according to claim 8, wherein the gaseous fluid is physiologically harmless and biocompatible, preferably carbon dioxide.

11. The device according to claim 9, wherein the cleaning using the gaseous fluid can be controlled by the control unit continuously with fluid pulses at intervals each with a duration of at most 1000 ms or continuously.

12. The device according to claim 1, wherein a fluid outside the cleaning module can be conveyed by at least one pumping device or a gas source by means of a supply line into the body cavity and through a discharge line out of the body cavity and the device furthermore comprises a pressure sensor for measuring the intracorporeal pressure, wherein the control unit controls the intracorporeal pressure in an event-controlled and/or time-controlled manner at least during the duration of a cleaning by means of a control of the at least one pumping device or a control of a pressure regulator (overpressure valve) such that the intracorporeal pressure does not exceed a predetermined maximum limiting value.

13. The device according to claim 1, wherein the nozzle can be fixedly positioned at a predetermined distance relative to the optical window and/or further window so that the fluid jet is directed over the entire external geometry of the optical window and/or the further window.

14. The device according to claim 1, wherein the optical window and/or further windows is or are formed by an at least partially convex surface (113).

15. The device according to claim 1,
wherein the optical window is an endoscope and wherein the at least one illumination device comprises light-conducting fiber bundles, LEDs (light emitting diodes), OLEDs (organic LEDs), one or more other light sources, or combinations thereof;
wherein the illumination device and/or the cleaning module is integrally connected and/or is detachably connected to the endoscope; and
wherein the endoscope is selected from a group having the following image capture devices:
a camera, an optoelectronic recording system, a digital camera, a CMOS image sensor, or a CCD image sensor.

16. The device according to claim 15, wherein the at least one image capture device having at least one illumination device is designed to be insertable and exchangeable in a shaft, and
the control unit has a memory and a processor for image recognition and optics recognition in order to recognize the image capture device and to transmit stored cleaning parameters, depending on the recognized image capture device, to the cleaning module for cleaning activation.

17. The device according to claim 1, wherein the cleaning module is part of a kit; and wherein the kit further comprises:
a pressure sensor for measuring the intracorporeal pressure and/or at least one further illumination device having an orientation identical or modified to that of the illumination device of the image capture device.

18. The device according to claim 17, wherein the kit comprises at least one partially circular or oval outer shell and/or a shaft.

19. A modular system comprising a device according to claim 1, the modular system furthermore comprising a shaft having a proximal and a distal end and at least one receptacle for at least one further component, wherein the component is selected from the following group comprising:
at least one working channel extending through the shaft from proximal to distal; and/or;
at least one suction channel extending through the shaft;
at least one flushing channel extending through the shaft;
at least one detachable distal sealing unit for sealing a fluid channel or a suction channel;
at least one fixed or detachable coupling to the image capture device;
at least one distal outlet or inlet which is aligned in the direction of the distal end or another predetermined object; and
combinations thereof.

20. The modular system according to claim 19, wherein the cleaning module and/or the components are reprocessable, sterilizable, or designed as disposable items.

21. The modular system according to claim 19, wherein the cleaning module and/or the components are detachably or integrally connected to another component and/or the image capture device.

22. The modular system according to claim 19, wherein the outer diameter of the shaft is configured such that the image capture device having at least one illumination device and having the cleaning module is receivable by an access system to the body cavity, preferably by a trocar sleeve.

23. A method for image analysis and/or cleaning of at least one window arranged at the distal end of an endoscope, comprising the following method steps:
providing an optical window of an image capture device and/or a further window for illuminating an object space using an illumination device and preferably at least one cleaning module;
capturing image data by means of the image capture device;
analyzing the image data by way of a control unit to detect a deterioration of the image quality, and on the basis of the detected image quality, automatically by means of a self-learning module and/or manually by the operator, outputting control instructions from the control unit to a unit of the endoscope to activate image optimization.

24. The method according to claim 23; wherein, on the basis of the method step of analyzing the image data and based on a deterioration in the image quality of the acquired image data, control instructions are output automatically or manually by an operator and/or at predetermined time intervals from the control unit to the cleaning module for activating a fluid pulse for cleaning and/or drying the optical window and/or the further window for image optimization, and
wherein the control unit sets at least one cleaning parameter which is selected from the group comprising:
pulse duration, number of pulses, pulse-pause ratio, total cleaning duration, type of fluid, fluid volume, fluid volumes, fluid velocity, and/or pressure.
